(19) United States
(12) Patent Application Publication
(10) Pub. No.: US 2003/0040476 A1
(43) Pub. Date: Feb. 27, 2003

(54) EAG gene

(76) Inventors: Walter Stuhmer, Gottingen (DE); Luis Pardo, Gottingen (DE); Rudiger Weseloh, Koln (DE)

Correspondence Address: FISH & NEAVE, 1251 AVENUE OF THE AMERICAS, 50TH FLOOR, NEW YORK, NY, 10020-1105, US

(21) Appl. No.: 10/128,323

(22) Filed: Apr. 22, 2002

Related U.S. Application Data
(63) Continuation of application No. PCT/EP00/10371, filed on Oct. 20, 2000.

(30) Foreign Application Priority Data
Oct. 20, 1999 (DE) EP 99120784.6

(51) Int. Cl. A61K 38/17; G01N 33/574; C07H 21/04; C12P 21/02; C12N 5/06

(52) U.S. Cl. 514/12; 435/69.1; 435/320.1; 435/325; 530/350; 536/23.5; 435/252.3; 435/254.2; 435/419

(57) ABSTRACT

The present invention relates to a novel human K⁺ ion channel, to nucleic acid molecules encoding the same and to vectors comprising said nucleic acid molecules. The invention additionally relates to antibodies specifically directed to the novel K⁺ ion channel and to phamaceutical compositions and diagnostic kits containing at least one of the above-mentioned components. Furthermore, the present invention relates to methods of treating a disease caused by malfunction of the polypeptide of the present invention or by the (over)expression of the nucleic acid molecule of the invention comprising administering an inhibitor of said (over)expression or of ion channel function or an inhibitor abolishing said malfunction to a patient in need thereof. Methods of devising drugs for treating or preventing the above-mentioned disease, methods of inhibiting cell proliferation and methods of prognosing cancer are additional embodiments comprised by the present invention. The invention also envisages specific antisense or gene therapies on the basis of the nucleic acid molecule of the invention for inhibiting undesired cellular proliferation, for example, in connection with cancer or in neurodegenerative diseases

Increasing evidence has accumulated showing the involvment of K⁺ channels in cell cycle and proliferation (see for example Bianchi, Cancer Res. 58 (1998), 815-822; DeCoursey, Nature 307 (1984), 465-468; Mauro, J. Invest. Dermatol. 108 (1997), 864-870; Nilius, J. Physiol. 445 (1992), 537-548; Pappas, Glia 22 (1998), 113-120; Pappone, Am. J. Physiol. 264 (1993), C1014-C1019; Skryma, Prostate 33 (1997), 112-122; Strobl, Gen. Pharmac. 26 (1995), 1643-1649; Woodfork, J. Cell. Physiol. 162 (1995), 163-171). Two mechanisms have been proposed to explain the role of K⁺ channels; they either influence the intracellular Ca²⁺ concentration (Santella, Biochem. Biophys. Res. Comm. 244 (1998), 317-324), or cell volume (Rouzaire-Dubois, J. Physiol. 510 (1998), 93-102). Both mechanisms would indirectly influence cell proliferation. The modulation of the ether àgogo (EAG) potassium channel during cell cycle-related events has previously been described (Brüggemann, Proc. Natl. Acad. Sci. USA 94 (1997), 537-542; Pardo, J. Cell. Biol. 143 (1998), 767-775; Pardo, EMBO J. 18 (1999), 101-108). The K⁺ current is inhibited following activation of cyclin-dependent kinases due to a voltage-dependent sodium block, which is not apparent in all phases of the cell cycle. It is still to be determined whether EAG, in addition to being regulated by the cell cycle, is also able to directly influence cell proliferation and growth.

The recently characterized potassium channel EAG (in the following here referred to as EAG1) was shown to have oncogenic properties (Pardo, (1999) loc. cit.). The expression of this EAG1 is strongly regulated during cell cycle which is related to its ability to control cell proliferation, since

(1) overexpression of EAG1 induces malignant transformation, as shown by faster growth, loss of contact inhibition, of substratum dependence and of growth factor dependence;

(2) EAG1 is preferentially expressed in human brain, but also in tumor cell lines from several origins (breast cancer, cervix cancer, neuroblastoma, melanoma) where the ectopic expression is at least permissive for the abnormal growth. Block of EAG1 expression leads to slower proliferation of these tumor cell lines; and

(3) in immune-deficient mice, implantation of tumor cells expressing EAG1 results in the growth of tumors much bigger and more aggressive than when wild type cells, or cells expressing a different potassium channel are implanted.

These findings demonstrate the direct influence of EAG1 activity on cell cycle and proliferation in the above-mentioned cells or tissues where EAG1 is expressed. However, it was not known whether other potassium channels with comparable activities exist and are expressed in these cells or tissues and/or in different cells or tissues. Such potassium channels could be used to enhance the degree of certainty of a diagnosis based on EAG1 expression. Thus, the technical problem underlying the present invention was to identify other potassium channels with comparable activities as EAG1 but a deviating tissue distribution of its expression.

The solution to said technical problem is achieved by providing the embodiments characterized in the claims.

Accordingly, the present invention relates to a nucleic acid molecule comprising a nucleic acid molecule encoding a (poly)peptide having a function of the human K⁺ ion hEAG2 channel which is

(a) a nucleic acid molecule comprising a nucleic acid molecule encoding the polypeptide having the amino acid sequence of SEQ ID: No 2;

(b) a nucleic acid molecule comprising the nucleic acid molecule having the DNA sequence of SEQ ID: No 1;

(c) a nucleic acid molecule hybridizing to the complementary strand of a nucleic acid molecule of (a) or (b); or

(d) a nucleic acid molecule being degenerate to the sequence of the nucleic acid molecule of (c).

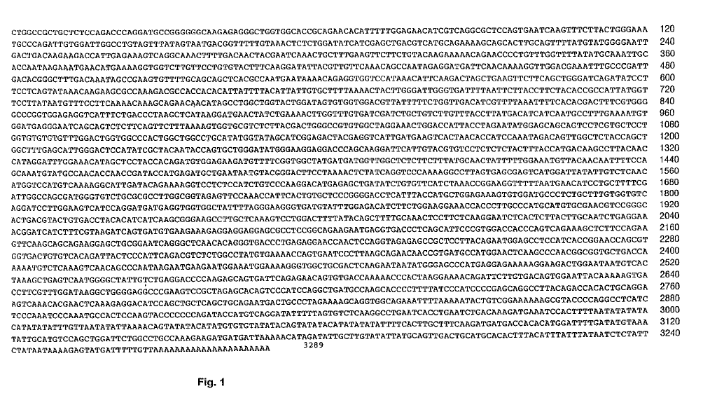

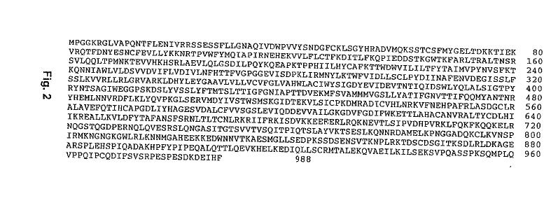

The nucleic acid molecule of the invention encodes a (poly)peptide which is or comprises homologues of the EAG1 channel. In this regard the term “a nucleic acid molecule comprising a nucleic acid molecule encoding a (poly)peptide having a function of the human K⁺ ion hEAG2 channel” may mean that said first mentioned nucleic acid molecule solely encodes said (poly)peptide. Thus, it may be identical to said second mentioned nucleic acid molecule. Alternatively, it may comprise regulatory regions or other untranslated regions. In a further embodiment, said first mentioned nucleic acid may comprise heterologous nucleic acid which may encode heterologous proteinaceous material thus giving rise, e.g., to fusion proteins. The DNA sequence of the hEAG2 cDNA clone isolated from a human brain library is shown by FIG. 1 (SEQ ID NO: 1) and the deduced protein sequence is shown in FIG. 2 (SEQ ID NO: 2). The terms “nucleic acid molecule”, “nucleic acid” and “polynucleotide” are used interchangeably herein.

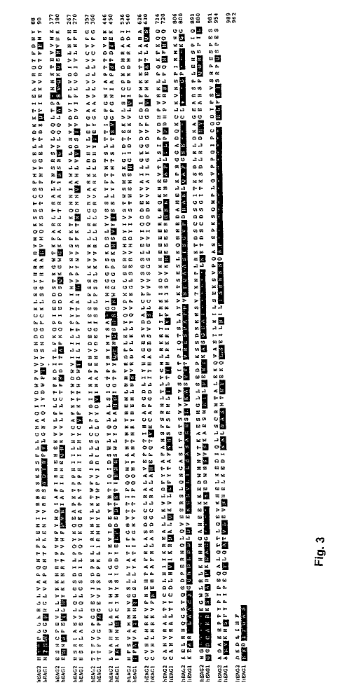

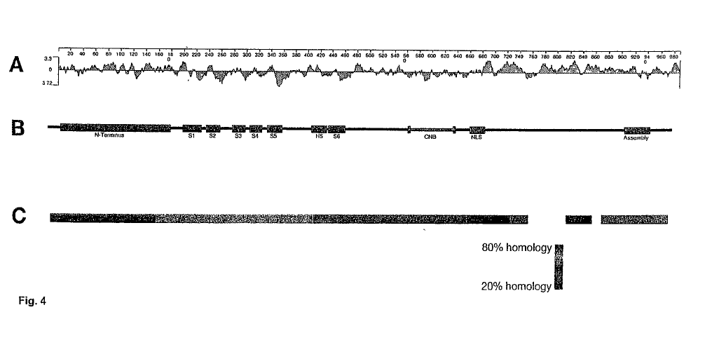

The main overall structural features of hEAG2 are conserved with EAG1. It consists of an N-terminal domain with the characteristic so-called eag-domain (Cabral,Cell 95 (1998), 649-655), six transmembrane segments (S1-S6) with S4 bearing abundant positive charges typical of the voltage-sensor, and the loop linking S5 and S6 (the main pore-lining region) highly conserved with respect to EAG1, a cyclic-nucleotide binding domain, a bipartite nuclear targeting sequence, and a subunit interaction domain (FIG. 4). However, the regions between these domains are poorly conserved. FIG. 3 shows an alignment between hEAG2 and EAG1.

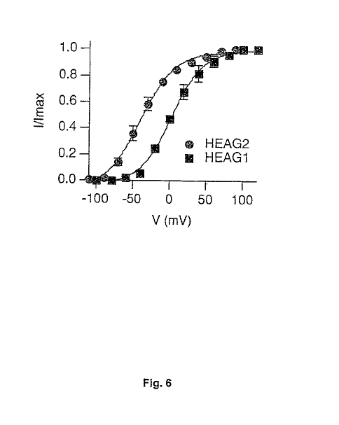

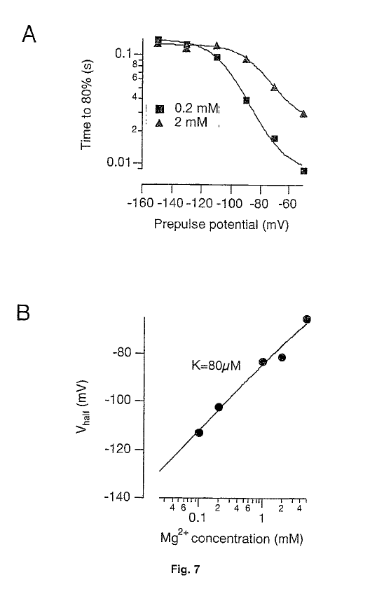

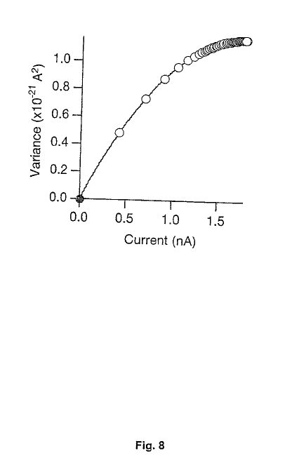

The term “having a function of a human K⁺ ion hEAG2 channel”, as used in connection with the present invention, has the following meaning: The channel has a single channel conductance in asymmetrical potassium, at 0 mV of about 8 pS (FIG. 8). This value clearly distinguishes the hEAG2 channel from the EAG1 channel for which a value of about 6 pS was measured as well as from the rat channel reag having a value of about 7 pS. In addition or in the alternative, the above term may have the following meaning: When measuring voltage-dependence of activation in high extracellular potassium using a two-electrode voltage-clamp it was found that in a conductance-voltage plot, the voltage for half-activation is shifted by about 40 mV to more negative values in the hEAG2 channel with respect to the EAG1 channel (See FIG. 6). Further, both EAG1 and hEAG2 show a time constant of activation highly dependent on the membrane potential before the stimulus. The more hyperpolarized the membrane is before the stimulus the slower is the activation of the channel. In addition, this effect is strongly modulated by extracellular Mg²⁺ in EAG1, but, surprisingly this has a much smaller effect in hEAG2. The apparent EC50 for the effect of magnesium in hEAG2 is very low (80 μM), but the overall effect of Mg²⁺ is not very dramatic. With a prepulse potential of −60 mV the activation in the presence of 2 mM Mg²⁺ is only three times slower than in the presence of 200 μM Mg²⁺ (FIG. 7). Thus, the electrophysiological characteristics of hEAG2 are readily distinguishable from those of EAG1 indicating different functional properties of hEAG2 and EAG1. On the basis of the above features, either alone or in combination, a differentiation based on function between the hEAG2 ion channel of the invention and the prior art channels, in particular of the EAG1 ion channel, is possible for the person skilled in the art without further ado. Preferably, the channel has all recited functions. The above values refer to values that are obtainable with the experimental set-up described in this specification. Alterations of experimental parameters such as the employment of a different expression system may, as is well known to the person skilled in the art, also change the above values. Yet, these embodiments are also comprised by the scope of the present invention.

The term “hybridizing” as used in accordance with the present invention relates to stringent or non-stringent hybridization conditions. Preferably, it relates to stringent conditions. Said hybridization conditions may be established according to conventional protocols described, for example, in Sambrook, “Molecular Cloning, A Laboratory Manual”, Cold Spring Harbor Laboratory (1989) N.Y., Ausubel, “Current Protocols in Molecular Biology”, Green Publishing Associates and Wiley Interscience, N.Y. (1989), or Higgins and Hames (eds) “Nucleic acid hybridization, a practical approach” IRL Press Oxford, Washington D.C., (1985). Hybridizing molecules or molecules falling under alternative (d), supra, also comprise fragments of the molecules identified in (a) or (b) wherein the nucleotide sequence need not be identical to its counterpart in SEQ ID NO: 1, said fragments having a function as indicated above.

An example of one such stringent hybridization condition is hybridization at 4× SSC at 65° C., followed by a washing in 0.1× SSC at 65° C. for one hour. Alternatively, an exemplary stringent hybridization condition is in 50% formamide, 4× SSC at 42° C. Examples of such non-stringent hybridization conditions are 4× SSC at 50° C. or hybridization with 30-40% formamide at 42° C. Complementary strands of hybridizing molecules comprise those which encode fragments, analogues or derivatives of the polypeptide of the invention and differ, for example, by way of amino acid and/or nucleotide deletion(s), insertion(s), substitution(s), addition(s) and/or recombination(s) or any other modification(s) known in the art either alone or in combination from the above-described amino acid sequences or their underlying nucleotide sequence(s).

Using the PESTFIND program (Rogers, Science 234 (1986), 364-368), PEST sequences (rich in proline, glutamic acid, serine, and threonine) can be identified, which are characteristically present in unstable proteins. Such sequences may be removed from the polypeptide of the invention in order to increase the stability and optionally the activity of the proteins. Methods for introducing such modifications in the nucleic acid molecules according to the invention are well-known to the person skilled in the art. The invention also relates to nucleic acid molecules the sequence of which differs from the nucleotide sequence of any of the above-described nucleic acid molecules due to the degeneracy of the genetic code. All such fragments, analogues and derivatives encoding the protein of the invention are included within the scope of the present invention, as long as the essential characteristic immunological and/or biological properties as defined above remain unaffected in kind, that is the novel nucleic acid molecules of the invention include all nucleotide sequences encoding proteins or peptides which have at least a part of the primary structural conformation for one or more epitopes capable of reacting with antibodies to said polypeptide which are encoded by a nucleic acid molecule as set forth above and which have comparable or identical characteristics in terms of biological activity. Part of the invention is therefore also concerned with nucleic acid molecules encoding a polypeptide comprising at least a functional part of the above identified polypeptide encoded by a nucleic acid sequence comprised in a nucleic acid molecule according to the invention.

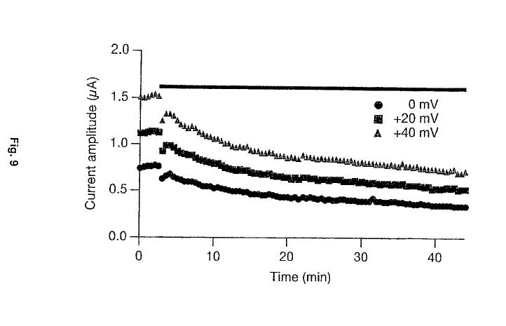

The most peculiar property of the EAG family, the cell cycle dependence, is present in hEAG2. Such modulation is radically different from the one of EAG1 in qualitative terms. hEAG1 responds to the progression of the cell cycle with a change in voltage dependence. This has been established by inducing the progression from G2 to M phase of meiosis I in Xenopus oocytes expressing hEAG2 by incubation with progesterone. For EAG1, the current obtained in M phase at +100 mV is less than the one obtained at +80 mV (a phenomenon termed rectification). After the rectification has been established, the current amplitude diminishes at all voltages. The rectification observed in EAG1 is not obvious in hEAG2, and only the reduction of current amplitude is detectable in the moment of progression from G2 to M phases of cell cycle. FIG. 9 shows the current amplitude measured during the progesterone treatment at three different voltages. The three traces diminish parallely at 0, +20 and +40 mV.

As expected from the strong cell-cycle dependent modulation, the overexpression of hEAG2 in CHO cells induces morphological changes, the most spectacular being the alteration of cell adhesion and contact inhibition, that results in the formation of visible cell clusters.

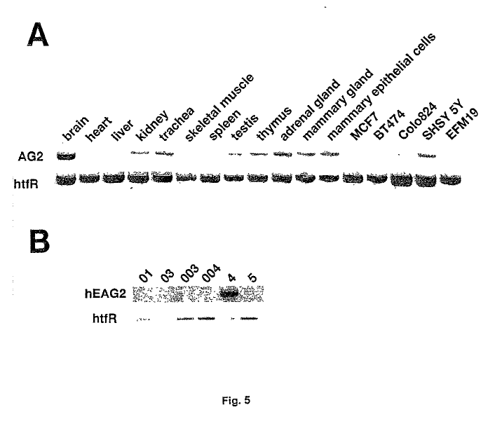

The tissue distribution of hEAG2 is radically different from that of EAG1. hEAG2 is expressed in brain, but also in heart, kidney, skeletal muscle, smooth muscle (trachea), spleen, testis, thymus, adrenal and mammary gland, and in several human cell lines (FIG. 5).

The chromosomal localization of hEAG2 was determined by FISH. hEAG2 is located on chromosome 14 (14q22-24).

When transfected into CHO cells, hEAG2 introduces very strong morphological modifications on the cells. Differences in the rate of growth as determined by quantifiable properties, such as metabolic activity or rate of DNA synthesis, were not detectable. Cells expressing hEAG2 are unable to form tumors when subcutaneously implantated into SCID mice.

The expression of hEAG2 in primary tumors was determined. With one exception, the expression levels were not significantly different from those of non-tumoral tissue. Since EAG1 was robustly expressed in 75% of those tumors, the expression of both genes must be independent. Similarly, in a screening of prostate tumors, hEAG2 was absent from all samples, while EAG1 was detected in 60%. Thus, in combination with EAG1, hEAG2 represents a useful tool for the characterization of the tumors, since its regulation seems to be maintained when that of EAG1 has been lost.

It is therefore possible to improve EAG1-based tumor diagnoses, preferably those which are based on the absence of EAG1 expression, by way of using the level of EAG2 expression as positive control.

In a preferred embodiment of the nucleic acid molecule of the invention, said nucleic acid molecule is DNA, such as genomic DNA. Whereas the present invention also comprises synthetic or semi-synthetic DNA molecules or derivatives thereof, such as peptide nucleic acid, the most preferred DNA molecule of the invention is cDNA.

In a further preferred embodiment of the present invention, said nucleic acid molecule is RNA, preferably mRNA.

Another preferred embodiment of the nucleic acid molecule of the invention encodes a fusion protein. For example, the nucleic acid molecule of the invention can be fused in frame to a detectable marker such as FLAG or GFP.

The invention further relates to a vector, particularly plasmid, cosmids, viruses and bacteriophages comprising the nucleic acid molecule of the invention. Such vectors may comprise further genes such as marker genes which allow for the selection of said vector in a suitable host cell and under suitable conditions. Thus the polynucleotide of the invention can be operatively linked in said vector to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40- , RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL). Preferably, said vector is an expression vector and/or a gene transfer or targeting vector. Expression vectors and gene targeting or transfer vectors are well-known in the art and can be adapted for specific purposes of the invention by the person skilled in the art. Thus, expression vectors derived from viruses such as retroviruses, vaccinia virus, adeno-associated virus, herpes viruses, or bovine papilloma virus, may be used for delivery of the polynucleotides or vectors of the invention into targeted cell populations. Methods which are well known to those skilled in the art can be used to construct recombinant viral vectors; see, for example, the techniques described in Sambrook, Molecular Cloning, A Laboratory Manual, Cold Spring Harbor Laboratory (1989) N.Y. and Ausubel, Current Protocols in Molecular Biology, Green Publishing Associates and Wiley Interscience, N.Y. (1989). Alternatively, the polynucleotides and vectors of the invention can be reconstituted into liposomes for delivery to target cells.

The invention furthermore relates to a host transformed with the vector of the invention. Said host may be a prokaryotic or eukaryotic cell. The polynucleotide or vector of the invention which is present in the host cell may either be integrated into the genome of the host cell or it may be maintained extrachromosomally. In this respect, it is also to be understood that the recombinant DNA molecule of the invention can be used for “gene targeting” and/or “gene replacement”, for restoring a mutant gene or for creating a mutant gene via homologous recombination; see for example Mouellic, Proc. Natl. Acad. Sci. USA, 87 (1990), 4712-4716; Joyner, Gene Targeting, A Practical Approach, Oxford University Press. Preferably, the host is a mammalian cell, a fungal cell, a plant cell, an insect cell or a bacterial cell. Preferred fungal cells are, for example, those of the genus Saccharomyces, in particular those of the species S. cerevisiae. The term “prokaryotic” is meant to include all bacteria which can be transformed or transfected with a polynucleotide for the expression of the protein of the present invention. Prokaryotic hosts may include gram negative as well as gram positive bacteria such as, for example, E. coli, S. typhimurium, Serratia marcescens and Bacillus subtilis. Methods for preparing fused, operably linked genes and expressing them in bacteria or animal cells are well-known in the art (Maniatis, et al., Molecular Cloning: A Laboratory Manual, Cold Spring Harbor Laboratory, Cold Spring Harbor, N.Y., 1989). The genetic constructs and methods described therein can be utilized for expression the protein of the present invention in prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. The transformed prokaryotic hosts can be grown in fermentors and cultured according to techniques known in the art to achieve optimal cell growth. The polypeptides of the invention can then be isolated from the grown medium, cellular lysates, or cellular membrane fractions. The isolation and purification of the microbially or otherwise expressed polypeptides of the invention may be by any conventional means such as, for example, preparative chromatographic separations and immunological separations such as those involving the use of monoclonal or polyclonal antibodies. As regards mammalian cells, HEK 293, CHO, HeLa and NIH 3T3 are preferred. As regards insect cells, it is most preferred to use Spodoptera frugiperda cells, whereas the most preferred bacterial cells are E. coli cells.

The invention also relates to a method of producing the (poly)peptide encoded by the nucleic acid molecule of the invention comprising culturing the host of the invention and isolating the produced (poly)peptide.

Depending on the vector construct employed, the (poly)peptide of the invention may be exported to the culture medium or maintained within the host. Suitable protocols for obtaining the (poly)peptide produced are well-known in the art for both ways of (poly)peptide production.

The present invention furthermore relates to a (poly)peptide encoded by the nucleic acid molecule of the invention or produced by the method of the invention. The new channel is envisaged to show a structure having a short amino-terminal region, probably intracellular, five membrane-spanning segments, a hydrophobic hairpin entering the membrane, a sixth transmembrane segment, and a long C-terminal cytoplasmic part comprising a cyclic-nucleotide binding consensus sequence, a nuclear localization consensus sequence, and a hydrophobic domain probably forming a coiled-coil structure. The polypeptide of the invention may also be a functional fragment of the hEAG2 K⁺ ion channel. By “functional fragment” polypeptides are meant that exhibit any of the activities of hEAG2 as described above. Using recombinant DNA technology, fragments of the (poly)peptide of the invention can be produced. These fragments can be tested for the desired function, for example, as indicated above, using a variety of assay systems such as those described in the present invention. Preferably, said fragments comprise the C-terminal portion of the novel ion channel.

The present invention also relates to an antibody specifically directed to the (poly)peptide of the invention. The antibody of the invention specifically discriminates between the hEAG2 channel and the prior art channels such as mouse and rat eag and preferably binds to epitopes in the C-terminal part of the ion channel. The term “antibody”, as used in accordance with the invention, also relates to antibody fragments or derivatives such as F(ab)₂, Fab′, Fv or scFv fragments; see, for example, Harlow and Lane, “Antibodies, A Laboratory Manual”, CSH Press 1988, Cold Spring Harbor, N.Y. Preferably, the antibody of the invention is a monoclonal antibody.

The invention also relates to a pharmaceutical composition comprising the nucleic acid molecule of the invention, the vector of the invention, the polypeptide of the invention and/or the antibody of the invention and a pharmaceutically acceptable carrier and/or diluent and/or excipient.

Examples of suitable pharmaceutical carriers and diluents as well as of excipients are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the patient in need thereof at a suitable dose. Administration of the suitable compositions may be effected by different ways, e.g., by oral, intravenous, intraperitoneal, subcutaneous, intramuscular, topical or intradermal administration. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10⁶ to 10¹² copies of the DNA molecule. The compositions of the invention may be administered locally or systemically. Administration will generally be parenterally, e.g., intravenously; DNA may also be administered directly to the target site, e.g., by biolistic delivery to an internal or external target site or by catheter to a site in an artery.

It is envisaged by the present invention that the various polynucleotides and vectors of the invention are administered either alone or in any combination using standard vectors and/or gene delivery systems, and optionally together with a pharmaceutically acceptable carrier or excipient. Subsequent to administration, said polynucleotides or vectors may be stably integrated into the genome of the subject. On the other hand, viral vectors may be used which are specific for certain cells or tissues and persist in said cells or tissues. Suitable pharmaceutical carriers and excipients are, as has been stated above, well known in the art. The pharmaceutical compositions prepared according to the invention can be used for the prevention or treatment or delaying of different kinds of diseases, which are related to the undesired (over)expression of the above identified nucleic acid molecule of the invention. In a preferred embodiment the pharmaceutical composition comprises antisense oligodesoxynucleotides specifically hybridizing to the nucleic acid molecules of the present invention, capable of regulating, preferably decreasing heavy expression.

Furthermore, it is possible to use a pharmaceutical composition of the invention which comprises the polynucleotide or vector of the invention in gene therapy. Suitable gene delivery systems may include liposomes, receptor-mediated delivery systems, naked DNA, and viral vectors such as herpes viruses, retroviruses, adenoviruses, and adeno-associated viruses, among others. Gene therapy, which is based on introducing therapeutic genes, for example for vaccination into cells by ex-vivo or in-vivo techniques is one of the most important applications of gene transfer. Suitable vectors, methods or gene-delivery systems for in-vitro or in-vivo gene therapy are described in the literature and are known to the person skilled in the art; see, e.g., Giordano, Nature Medicine 2 (1996), 534-539; Schaper, Circ. Res. 79 81996), 911-919; Anderson, Science 256 (1992), 808-813; Isner, Lancet 348 (1996), 370-374; Muhlhauser, Circ. Res. 77 (1995), 1077-1086; Onodera, Blood 91 (1998), 30-36; Verzeletti, Hum. Gene Ther. 9 (1998), 2243-2251; Verma, Nature 389 (1997), 239-242; Anderson, Nature 392 (Supp. 1998), 25-30; Wang, Gene Therapy 4 (1997), 393-400; Wang, Nature Medicine 2 (1996), 714-716; WO 94/29469; WO 97/00957; U.S. Pat. Nos. 5,580,859; 5,589,466; 4,394,448 or Schaper, Current Opinion in Biotechnology 7 (1996), 635-640, and references cited therein. The nucleic acid molecules and vectors of the invention may be designed for direct introduction or for introduction via liposomes, or viral vectors (e.g. adenoviral, retroviral) into the cell. Additionally, a baculoviral system can be used as eukaryotic expression system for the nucleic acid molecules of the invention. Delivery of nucleic acids to a specific site in the body for gene therapy may also be accomplished using a biolistic delivery system, such as that described by Williams (Proc. Natl. Acad. Sci. USA 88 (1991), 2726-2729).

Standard methods for transfecting cells with recombinant DNA are well known to those skilled in the art of molecular biology, see, e.g., WO 94/29469. Gene therapy may be carried out by directly administering the recombinant DNA molecule or vector of the invention to a patient or by transfecting cells with the polynucleotide or vector of the invention ex vivo and infusing the transfected cells into the patient. Furthermore, research pertaining to gene transfer into cells of the germ line is one of the fastest growing fields in reproductive biology. Gene therapy, which is based on introducing therapeutic genes into cells by ex vivo or in vivo techniques is one of the most important applications of gene transfer. Suitable vectors and methods for in vitro or in vivo gene therapy are described in the literature and are known to the person skilled in the art; see, e.g., WO94/29469, WO 97/00957 or Schaper (Current Opinion in Biotechnology 7 (1996), 635-640) and references cited above. The polynucleotides and vectors comprised in the pharmaceutical composition of the invention may be designed for direct introduction or for introduction via liposomes, or viral vectors (e.g. adenoviral, retroviral) containing said recombinant DNA molecule into the cell. Preferably, said cell is a germ line cell, embryonic cell, stem cell or egg cell or derived therefrom. An embryonic cell can be for example an embryonic stem cell as described in, e.g., Nagy, Proc. Natl. Acad. Sci. USA 90 (1993) 8424-8428.

It is to be understood that the introduced polynucleotides and vectors of the invention express the (poly)peptide of the invention after introduction into said cell and preferably remain in this status during the lifetime of said cell. For example, cell lines which stably express the polynucleotide under the control of appropriate regulatory sequences may be engineered according to methods well known to those skilled in the art. Rather than using expression vectors which contain viral origins of replication, host cells can be transformed with the polynucleotide or vector of the invention and a selectable marker, either on the same or separate vectors. Following the introduction of foreign DNA, engineered cells may be allowed to grow for 1-2 days in an enriched media, and then are switched to a selective medium. The selectable marker in the recombinant plasmid confers resistance to the selection and allows for the selection of cells having stably integrated the plasmid into their chromosomes and grow to form foci which in turn can be cloned and expanded into cell lines. Such engineered cell lines are particularly useful in screening methods or methods for identifying an inhibitor of the polypeptide of the present invention as described below.

A number of selection systems may be used, including but not limited to the herpes simplex virus thymidine kinase (Wigler, Cell 11(1977), 223), hypoxanthine-guanine phosphoribosyltransferase (Szybalska, Proc. Natl. Acad. Sci. USA 48 (1962), 2026), and adenine phosphoribosyltransferase (Lowy, Cell 22 (1980), 817) in tk, hgprt or aprt cells, respectively. Also, antimetabolite resistance can be used as the basis of selection for dhfr, which confers resistance to methotrexate (Wigler, Proc. Natl. Acad. Sci. USA 77 (1980), 3567; O'Hare, Proc. Natl. Acad. Sci. USA 78 (1981), 1527), gpt, which confers resistance to mycophenolic acid (Mulligan, Proc. Natl. Acad. Sci. USA 78 (1981), 2072), neo, which confers resistance to the aminoglycoside G-418 (Colberre-Garapin, J. Mol. Biol. 150 (1981), 1), hygro, which confers resistance to hygromycin (Santerre, Gene 30 (1984), 147), Shble, which confers resistance to Zeocin® (Mulsant, Somat. Cell. Mol. Genet. 14 (1988), 243-252) or puromycin (pat, puromycin N-acetyl transferase). Additional selectable genes have been described, for example, trpB, which allows cells to utilize indole in place of tryptophan; hisD, which allows cells to utilize histinol in place of histidine (Hartman, Proc. Natl. Acad. Sci. USA 85 (1988), 8047); and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.). Cells to be used for ex vivo gene therapy are well known to those skilled in the art. For example, such cells include for example cancer cells present in blood or in a tissue or preferably the corresponding stem cells.

Furthermore, the invention relates to a diagnostic composition comprising the nucleic acid molecule of the invention, the vector of the invention, the polypeptide of the invention and/or the antibody of the invention.

The diagnostic composition of the invention is useful in detecting the onset or progress of diseases related to the undesired lack of expression, expression or overexpression of the nucleic acid molecule of the invention. As has been pointed out herein above, such diseases are interrelated or caused by an increased or ongoing cellular proliferation. Accordingly, the diagnostic composition of the invention may be used for assessing the onset or the disease status of cancer. Having thus an early criterium for tumor activity, suitable counter-measures can immediately be applied. Such an immediate action will, of course, significantly improve the prognosis of the patient. These considerations equally apply to the diagnosis of metastases and recurrent tumors.

On the other hand, not all types of tumors may be characterized by an undesired lack of expression, expression or overexpression of the nucleic acid molecule of the invention. Alternatively, said lack of expression or (over)expression may occur only in certain stages, such as early stages, of tumor development. Therefore, the diagnostic composition of the invention may also or alternatively be employed as a means for the classification of tumors or of the developmental status of a tumor.

Additionally, one major goal of the diagnostic composition of the present invention is to assist in diagnostic methods which are based on the measurement of EAG1 expression (Pardo, EMBO J. 18 (1999), 101-108 and WO 99/54463), preferably in methods of diagnosing tumors. Such applications refer to tissues where hEAG2 shows an unaltered expression when comparing a tumor and the corresponding non-tumoral tissue. The expression level of hEAG2 may be taken as a control, preferably as a positive control, in order to calibrate measurements of EAG1 expression and thereby to improve the significance of EAG1 based diagnoses. Due to the structural and functional similarities of EAG1 and hEAG2 which are contrasted by their differing regulation of gene expression, hEAG2 is suited to serve as a “perfect control” for the specificity of any diagnostic method based on EAG1.

The present invention therefore also relates to a method of diagnosing tumors comprising

(a) determining the level of expression of EAG1 and hEAG2 in a sample of a subject; and

(b) diagnosing a predisposition to have and preferably diagnosing a tumor if the expression level of EAG1 is aberrant, whereby the expression level of hEAG2 is normal, i.e. corresponds to a level obtained with healthy tissue.

Naturally, the or most of the applications of the composition of the invention described here for tumors also apply to other diseases interrelated with or caused by the undesired (over)expression of the nucleic acid molecule of the invention. These applications and corresponding methods are also comprised by the invention where steps corresponding to the steps referred to above are carried out.

Furthermore, a disease as recited throughout this specification also could be caused by a malfunction of the polypeptide of the present invention. Said disease could be interrelated or caused by, for example, an increased or reduced gene dosis of the polypeptide of the present invention, an increased or reduced activity of said polypeptide e.g. due to a modification in the primary amino acid sequence as compared to the corresponding wild-type polypeptide in a cell or tissue or a loss of the regulation of the activity of said polypeptide. Said disease might further be caused by an incorrect expression of the polypeptide during cell cycle progression or cell development. For example, mutated binding sites to intracellular or extracellular compounds, e.g. ions or second messengers or regulatory proteins, might result in a malfunction of the polypeptide of the present invention as it changes the binding characteristics for said compounds regulating the activity of said polypeptide. Malfunction could also be caused by defective modifications sites, for example, phosphorylation or glycosylation sites. It also might be caused by incorrect splicing events and therefore by expression of a truncated or extended polypeptides, for example.

Thus, in a further embodiment the diagnostic composition described above could also be used to detect a malfunction of the polypeptide of the present invention.

The invention also relates to methods for preventing or treating a disease which is caused by the undesired expression or overexpression of the nucleic acid molecule of the invention, comprising introducing an inhibitor of the expression of the nucleic acid molecule of the invention or an inhibitor of the function of the (poly)peptide of the invention into a mammal affected by said disease or being suspected of being susceptible to said disease. The invention likewise relates to the use of such inhibitors for the production of a pharmaceutical composition for preventing or treating said disease. Methods for obtaining such inhibitors are described further below.

In another aspect the invention relates to methods for preventing or treating a disease which is caused by the undesired lack of expression of the nucleic acid molecule of the invention comprising introducing a nucleic acid molecule of the invention, the vector of the invention, the host of the invention or the (poly)peptide of the invention into a mammal affected by said disease or being suspected of being susceptible to said disease. The invention likewise relates to the use of said nucleic acid molecule, vector, host or (poly)peptide for the production of a pharmaceutical composition for preventing or treating said disease.

In a further embodiment, the invention relates to a method for preventing or treating a disease which is caused by the malfunction of the polypeptide of the invention, comprising introducing an inhibitor of the expression of the nucleic acid molecule of the present invention or an inhibitor or a modifying agent of the malfunction of the (poly)peptide of the present invention or a nucleic acid molecule coding hEAG2 or a polypeptide having hEAG2 activity into a mammal affected by said disease or being suspected of being susceptible to said disease. Methods for introduction of a nucleic acid molecule of the present invention encoding hEAG2 into a cell or subject, i.e. gene therapy, are described within this specification as well as methods for the identification of inhibitors of the expression of a nucleic acid molecule of the present invention. Furthermore, inhibitors or modifying agents of the malfunction of the polypeptide of the present invention can be identified according to methods for the identification of inhibitors inhibitors of the polypeptide of the present invention known to a person skilled in the art (see below). For example, some genetic changes causing a malfunction of the polypeptide of the present invention lead to altered protein conformational states. Mutant proteins could possess a tertiary structure that renders them far less capable of fascilitating ion transport. Restoring the normal or regulated conformation of mutated proteins is the most elegant and specific means to correct these molecular defects. Pharmacological manipulations thus may aim at restoration of wild-type conformation of the protein. Thus, the polynucleotides and encoded proteins of the present invention may also be used to design and/or identify molecules which are capable of activating the wild-type function of a derivative of the polypeptide of the present invention displaying said malfunction.

The doses and routes for the administration for the treatment of a patient in need thereof have already been discussed herein above in connection with the pharmaceutical composition of the invention. Diseases that may be treated using the method of the present invention comprise any diseases that are correlated with cellular proliferation. Preferred diseases that fall into this category are tumor diseases such as cancer (breast cancer, neuroblastoma etc.), psoriasis, and degenerative diseases, especially those of the nervous system such as Alzheimers disease, multiple sclerosis, lateral amyotrophic sclerosis, and Parkinson's disease.

Preferably, said inhibitor of the expression or overexpression of said nucleic acid molecule is a nucleic acid molecule of the invention that specifically hybridizes to the nucleic acid molecule encoding the ion channel of the invention or fragment thereof. In a preferred embodiment this nucleic acid molecule can be an antisense oligodesoxynucleotide (ODN).

In a further preferred embodiment, said inhibitor of polypeptide function is the antibody of the invention or a drug. Said drug can be histamine receptor H1 inhibitor. Preferably, said drug inhibits active hEAG2, for example, acts as use-dependent, probably open-channel blocker, preferably said drug is astemizole or terfenadine. Further suitable drugs can be identified or designed by the person skilled in the art on the basis of the teachings of the present invention. Preferably, the drug will have an affinity to the hEAG2 channel in the mM range, more preferable in the nM range or lower. Preferably, the drug has no effect on other channels, for example on cardiac channels.

In a further preferred embodiment of the invention, said method further comprises prior to the introduction step,

(a) obtaining cells from the mammal infected by said disease and, after said introduction step, wherein said introduction is effected into said cells,

(b) reintroducing said cells into said mammal or into a mammal of the same species.

This embodiment of the present invention is particularly useful for gene therapy purposes which will reduce the treatment duration largely and increase the effectivity and reduce (even eliminate) side effects. In addition, this embodiment of the method of the invention can also be employed in the context or in combination with conventional medical therapy. The removal from and the reintroduction into said mammal may be carried out according to standard procedures.

Preferably, the above referenced cell is a germ cell, an embryonic cell or an egg cell or a cell derived from any of these cells.

In a further embodiment, the present invention relates to a method for preventing and/or treating a congenital disease comprising introducing a nucleic acid molecule of the present invention, a vector of the present invention or a drug capable of reconstituting the function of a hEAG2 protein the activity of which is blocked or diminished into a mammal affected by said disease or being susceptible to said disease.

The present invention also relates to a method for diagnosing a congenital disease or a susceptibility to a congenital disease related to a malfunction of a hEAG2 protein of the present invention comprising determining a mutation in a nucleic acid sequence encoding said polypeptide.

Preferably, the above referenced congenital disease is arrythmogenic right ventricular cardiomyopathy (ARVC).

The invention further relates to a method of designing a drug for the treatment of a disease which is caused by the undesired lack of expression, or expression or overexpression of the nucleic acid molecule of the invention comprising:

(a) identification of a specific and potent drug;

(b) identification of the binding site of said drug by site-directed mutagenesis and chimeric protein studies;

(c) molecular modeling of both the binding site in the (poly)peptide and the structure of said drug; and

(d) modifications of the drug to improve its binding specificity for the (poly)peptide.

The term “specific and potent drug” as used herein refers to a drug that potently and specifically blocks hEAG2 function.

All techniques employed in the various steps of the method of the invention are conventional or can be derived by the person skilled in the art from conventional techniques without further ado. Thus, biological assays based on the herein identified features of the ion channel of the invention may be employed to assess the specificity or potency of the drugs wherein the decrease of one or more activities of the ion channel may be used to monitor said specificity or potency. Steps (b) and (d) can be carried out according to conventional protocols described, for example, in K. L. Choi, C. Mossman, J. Aubé & G. Yellen. The International Quaternary Ammonium Receptor Site of Shaker Potassium Channels. Neuron 10, 533-541 (1993), C.-C. Shieh & G. E. Kirsch: Mutational Analysis of Ion Conduction and Drug Binding Sites in the Inner Mouth of Voltage-Gated K⁺-Channels. Biophys. J. 67, 2316-2325 (1994), or C. Miller: The Charybdotoxin Family of K⁺-Channel-Blocking Peptide. Neuron 15, 5-10 (1995).

For example, identification of the binding site of said drug by site-directed mutagenesis and chimerical protein studies can be achieved by modifications in the (poly)peptide primary sequence that affect the drug affinity; this usually allows to precisely map the binding pocket for the drug.

As regards step (c), the following protocols may be envisaged: Once the effector site for drugs has been mapped, the precise residues interacting with different parts of the drug can be identified by combination of the information obtained from mutagenesis studies (step (b)) and computer simulations of the structure of the binding site (since a potassium channel has recently been crystallized in the art, this can now be done by the person skilled in the art without further ado) provided that the precise three-dimensional structure of the drug is known (if not, it can be predicted by computational simulation). If said drug is itself a peptide, it can be also mutated to determine which residues interact with other in the hEAG2 molecule.

Finally, in step (d) the drug can be modified to improve its binding affinity or its potency and specificity. If, for instance, there are electrostatic interactions between a particular residue of hEAG2 and some region of the drug molecule, the overall charge in that region can be modified to increase that particular interaction; additionally, if those interactions occur with a region of hEAG2 that is not conserved with other channel proteins, it is conceivable that an improvement of that interaction while other binding factors are weakened will improve the specificity of the drug.

Identification of binding sites may be assisted by computer programs. Thus, appropriate computer programs can be used for the identification of interactive sites of a putative inhibitor and the polypeptide of the invention by computer assisted searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. Modifications of the drug can be produced, for example, by peptidomimetics and other inhibitors can also be identified by the synthesis of peptidomimetic combinatorial libraries through successive chemical modification and testing the resulting compounds. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors of the polypeptide of the invention can be used for the design of peptidomimetic inhibitors, e.g., in combination with the (poly)peptide of the invention (Rose, Biochemistry 35 (1996), 12933-12944; Rutenber, Bioorg. Med. Chem. 4 (1996), 1545-1558).

The invention also relates to a method of identifying an inhibitor of the expression of the nucleic acid of the invention or of a function of the (poly)peptide of the invention comprising:

(a) testing a compound for the inhibition or reduction of translation wherein said compound is selected from antisense oligonucleotides and ribozymes; or

(b) testing a compound for the inhibition of transcription wherein said compound binds to the promoter region of the gene encoding the (poly)peptide of the invention and preferably with transcription factor responsive elements thereof; or

(c) testing peptides or antibodies suspected to block the proliferative activity of the (poly)peptide of the invention for said blocking activity.

As regards alternative (b) referred to above, it may be advantageous to first characterize the promoter region and locate transcription factor responsive sequences in it. Then it would be possible to genetically manipulate the promoter to render it more sensitive to repressors or less sensitive to enhancers.

Turning now to alternative (c), it may be advantageous to first locate the part or parts of the ion channel of the invention implicated in the generation of proliferation disorders. Compounds that have been positive in one of the test systems are, prima facie, useful as inhibitors.

Peptidomimetics, phage display and combinatorial library techniques are well-known in the art and can be applied by the person skilled in the art without further ado to the improvement of the drug or inhibitor that is identified by the basic method referred to herein above.

In a further aspect, the invention relates to a method of selecting a drug specifically inhibiting the expression or function of EAG1 while not effecting hEAG2 in tumor cells comprising

(a) testing a drug for inhibition of EAG1 expression or function;

(b) testing a drug for inhibition of the expression of the nucleic acid molecule of the invention or of the function of the polypeptide of the invention; and

(c) selecting a drug that tested positive in step (a) and negative in step (b).

This embodiment takes into account the great similarity between EAG1 and the protein of the present invention, hEAG2, which is described in detail herein above. As also already mentioned, EAG1 is a potent oncogene (Pardo, EMBO J. 18 (1999), 101-108) and a specific inhibitor of its expression or function is a promising candidate drug for treating tumors in which overexpression or malfunction of EAG1 is involved. Since, on the other hand, hEAG2 was often shown to have an unaltered expression in tumors compared to the corresponding non-tumoral tissue, it may in most instances not be desirable to affect hEAG2 function or expression with an inhibitor directed towards EAG1. Moreover, such an unspecific cross-reactivity could have detrimental consequences to the organism and might cause severe side effectes. Thus, a drug specifically inhibiting the expression or function only of EAG1 is in many cases necessary to ensure successful anti-tumor therapy.

EAG1 is for example described in Pardo (EMBO J. 18 (1999), 101-108) and WO 99/54463 as regards the encoding nucleotide sequence as well as protein function.

The person skilled in the art knows how to prepare potential drug compounds. Compounds that are suited to inhibit gene expression encompass nucleic acid molecules that specifically interact with the target nucleic acid molecule such as for example antisense molecules or ribozymes. Compounds that are suited to inhibit EAG1 protein function encompass antibodies or fragments or derivatives thereof or other protein binding molecules. Other protein inhibitors such as small organic compounds as well as peptides or modifications thereof as they are known in the art may as well be used in the method of the present embodiment. The methods of designing drugs which is described above in connection with the protein of the invention can also be applied for providing potential drug compounds to be tested in steps (a) and (b) of the present method. The testing steps correspond to those described above in connection with the method of identifying inhibitors.

Furthermore, the present invention relates to a method for the production of a pharmaceutical composition comprising the steps of the above-described methods for designing or selecting drugs or for identifying an inhibitor and, furthermore, the step of formulating said drug or inhibior identified, selected or identified in the prece drug steps in a pharmaceutically acceptable form.

The step of formulating a compound, such as a drug or inhibitor, in a pharmaceutically acceptable form so as to obtain a pharmaceutical composition of the present invention has already been described in detail above.

In a further embodiment, the present invention relates to a method of inhibiting cell proliferation comprising applying an inhibitor to expression of the nucleic acid of the invention or the (poly)peptide of the invention. The method of the invention may be carried out in vitro, ex vivo or when application is to a subject, in vivo.

The present invention also relates to a method of prognosing cancer and/or neurodegenerative diseases and/or psoriasis and/or a malfunction of the heart comprising assessing the expression of the nucleic acid molecule of the invention or assessing the quantitative presence of the (poly)peptide of the invention. In a preferred embodiment said cancer is a mamma carcinoma or neuroblastoma, in a more preferred embodiment said cancer is breast adenocarcinoma, breast carcinoma ductal type, or cervix carcinoma. In a further embodiment said neurodegenerative diseases is Alzheimer's disease, Parkinson's disease, lateral amytrophic sclerosis or multiple sclerosis.

The method of the invention may be carried out in vitro, in vivo, or ex vivo. Suitable protocols for carrying out the method of the invention are well-known in the art and include, as regards in vitro techniques, Northern blotting for the assessment of the level of mRNA or the analysis of tissue by microscopic techniques using, for example, antibodies that specifically recognize the (poly)peptide of the invention. One or more these techniques may be combined with PCR based techniques which may also or in combination with further (conventional) techniques be used for the above recited assessment.

In a preferred embodiment of the above-mentioned methods of the invention, said mammal is a human, rat or mouse.

The present invention further relates to the use of the nucleic acid molecules of the invention in gene therapy. As has been pointed out here above, gene therapy may be designed to inhibit cell proliferation and thus treat any disease affected thereby such as cancer or psoriasis in a specific way. The invention particularly envisages two independent lines carrying out such gene therapy protocols:

(a) Mutagenesis of the channel together with chemical engineering of HI antagonists (preferably of astemizole) in order to obtain a drug specific for hEAG2;

(b) Quantitative and qualitative analysis of the expression levels of hEAG2 in cancer tissue, in order to design a diagnostic and/or prognostic method.

This would also allow the design of genetic therapies against specific tumors.

For example, the nucleic acid molecule may be introduced in vivo into cells using a retroviral vector (Naldini et al., Science 272 (1996), 263-267; Mulligan, Science 260 (1993), 926-932) or another appropriate vector. Likewise, in accordance with the present invention cells from a patient can be isolated, modified in vitro using standard tissue culture techniques and reintroduced into the patient. Such methods comprise gene therapy or gene transfer methods which have been referred to herein above.

Finally, the present invention relates to a kit comprising the nucleic acid molecule specifically hybridizing to the nucleic acid molecule encoding the (poly)peptide of the invention, the vector of the invention, the polypeptide of the invention and/or the antibody of the invention.

The kit of the invention can, inter alia, be employed in a number of diagnostic methods referred to above. The kit of the invention may contain further ingredients such as selection markers and components for selective media suitable for the generation of transformed host cells. Furthermore, the kit may include buffers and substrates for reporter genes that may be present in the recombinant gene or vector of the invention. The kit of the invention may advantageously be used for carrying out the method of the invention and could be, inter alia, employed in a variety of applications referred to herein, e.g., in the diagnostic field or as research tool. The parts of the kit of the invention can be packaged individually in vials or in combination in containers or multicontainer units. Manufacture of the kit follows preferably standard procedures which are known to the person skilled in the art.

Several documents are cited throughout the text of this specification. Each of the documents cited herein (including any manufacturers specifications, instructions, etc.) are hereby incorporated herein by reference; however, there is no admission that any document cited is indeed prior art as to the present invention.

The figures show:

FIG. 1 cDNA sequence of the hEAG2 clone.

FIG. 2 Deduced amino acid sequence of hEAG2 in single letter code.

FIG. 3 Alignment between hEAG2 and EAG1 protein sequences. The shaded residues correspond to the sequence divergences.

FIG. 4A. Predicted hydropathy plot of hEAG2. B. Schematic representation of the domain distribution of hEAG2 (S1-S6: Transmembrane domains. H5: Pore region. CNBD: Cyclic nucleotide-binding domain. NLS: nuclear localization signal.). C. Color-coded representation of the homology between EAG1 and hEAG2.

FIG. 5A. RT-PCR on RNA from different tissues with primers specific for hEAG2. B. RT-PCR on RNA obtained from several breast tumors. Five of the tumors were negative, while #4 shows amplification of EAG2. Human transferrin receptor (htfR) signals are shown at the bottom.

FIG. 6 Voltage-dependence of hEAG2 current as compared to hEAG1. The conductance of the membrane was calculated using a tail current protocol in the presence of 115 mM kCl in the external solution. The error bars represent S.E.M. for 6 independent experiments.

FIG. 7 The activation of hEAG2 depends both on the voltage previous to the stimulus and the external magnesium concentration. A. Time required to achieve 80% of the maximal current amplitude when the membrane is maintained at different voltages between −150 and −50 mV before the stimulus, in the presence of 200 μM external MgCl₂ (squares) or 2 mM (triangles). The solid line is a fit to a Boltzmann equation from which V_{half} was calculated. B. Plot of V_{half} versus Mg²⁺ concentration. The apparent EC₅₀ was 80 μM.

FIG. 8 Variance vs. current plot obtained with hEAG2 expressing CHO cells. The plot has been obtained from 500 test pulses to +60 mV. The estimated single channel conductance was 7.97 pS.

FIG. 9 Reduction on current amplitude upon progression of the G2-M transition of the cell cycle. The Figure represents the amplitudes measured for three different voltages to show that there is no voltage-dependent blockade of the channel.

The examples illustrate the invention.

### EXAMPLE 1

### Cloning of the cDNA of the hEAG2 K⁺ ion Channel

Specific oligonucleotides to amplify hEAG2 cDNA from Marathon-cDNA of human total brain and human hippocampus—purchased from Clontech—were designed using the sequence of est clone c-0bf08 (Accession # F05455) as a template. The oligonucleotides had the following sequences:

| | |
|---|---|
| 5′-GGTTTCCTTCCAGAAGATGTCTCCAAATA-3′ | (SEQ ID NO:3) |
| 5′-GATGACTTCCAAGGATCCTGACACC-3′ | (SEQ ID NO:4) |
| 5′-CCAATGCAAAAGCAGGATGTTCATTAA-3′ | (SEQ ID NO:5) |

These oligonucleotides were used together with the RACE oligonucleotides AP1 or AP2 (Clontech). The RACE-PCR yielded DNA-fragments which were cloned into the pGEM-T vector (Promega) and sequenced. Another two rounds of RACE-PCRs, subsequent cloning and sequencing of the DNA fragments, were performed to generate the complete 5′-sequence of the hEAG2 open reading frame. The following oligonucleotides were used:

| | |
|---|---|
| 5′-AATCATCCTCTATTGGCTGTTTGAACAAC-3′ | (SEQ ID NO:6) |
| 5′-TAATATCCTTGAAAGTACACAGGAACAAG-3′ | (SEQ ID NO:7) |
| and | |
| 5′-CAGGCCAATCCACAATCTGGGCATTTC-3′ | (SEQ ID NO:8) |

These oligonucleotides were used together with the RACE oligonucleotides AP1 or AP2 (Clontech).

The cDNA coding for the complete open reading frame of hEAG2 was then cloned from human total brain and hippocampus RNA (Clontech). The cDNA was amplified in three fragments using RT-PCR. The oligonucletides for the amplification were:

5′-CTGGCCGCTGCTCTCCAGACC-3′ (SEQ ID NO:9) and 5′-TCACAAACCAAGTTTTCAGATAGTTCA-3′ (SEQ ID NO: 10) for the 5′-fragment of 910 bp, and 5′-AGAGTTCCAAACCATTCACTGTGCT-3′ (SEQ ID NO:111) and 5′-CCAGMTCCAGCTGGACATGCAATAT-3′ (SEQ ID NO:12) for the middle fragment of 1429 bp, and 5′-CAMGCAGMCMCATAGCCTGGCTG-3′ (SEQ ID NO:13) and 5′-GGTTTCCTTCCAGMGATGTCTCCAAATA-3′ (SEQ ID NO:14) for the 3′-fragment of 1145 bp.

Each of the three fragments was cloned into the pGEM-T vector and sequenced. The fragments were subsequently excised from the vectors using restriction enzymes EagI/ApaI, ApaI/BamHI and BamHI/NdeI, respectively. The cDNA fragments were isolated, ligated and amplified by PCR with following oligonucleotides:

5′-TATAGGTACCGAATTCGCGGCCGCCACCATGCCGGGGGGCMGAGA-3′ (SEQ ID NO:15) and

5′-TCTAGGAGCTCGAGTCTAGATTAAAAGTGGATTTCATCTTTGTC-3′ (SEQ ID NO:16).

The amplified cDNA fragment of 3015 bp was isolated, digested with the restriction enzymes Kpnl and Sacl, and subcloned into the pGEM-T vector.

### EXAMPLE 2

### Expression of hEAG2 in Different Human Tissues

500 ng of total RNA from different human tissues were reverse transcribed and amplified (RT-PCR) using the oligonucleotides

5′-ACCATGACMGCCTTACAACCATAGGA-3′ (SEQ ID NO:17) and 5′-GGTTTCCTTCCAGAAGATGTCTCCAAATA-3′ (SEQ ID NO:18).

The expression of hEAG2 could be detected in the RNAs from brain, heart, kidney, skeletal muscle, trachea, testis, thymus, adrenal gland, mammary gland and mammary epithelial cells (FIG. 5A). No expression could be detected in RNA from liver and spleen.

Using the same approach the expression of hEAG2 in different tumoral human cell lines was tested (FIG. 5A). Expression was found in the following cell lines: MCF-7 (breast adenocarcinoma), BT-474 (breast carcinoma, ductal type, from a solid tumor), COLO-824 (breast carcinoma, from pleural fluid), SHSY 5Y (neuroblastoma). No expression could be detected in the RNA of EFM-19 cells (breast carcinoma, ductal type, from pleural fluid).

In one of five different RNA samples from primary mammary tumors, the expression of hEAG2 could be detected in one sample using the oligonucleotides 5′-CAAAGCAGAACAACATAGCCTGGCTG-3′ (SEQ ID NO:19) and 5′-GGTTTCCTTCCAGAAGATGTCTCCAAATA-3′ (SEQ ID NO:20) for RT-PCR and the oligonucleotides 5′-GTACTGGATAGTGTGGTGGACGTTAT-3′ (SEQ ID NO:21) and 5′-GATGACTTCCAAGGATCCTGACACC-3′ (SEQ ID NO:22) for a subsequent nested-PCR (FIG. 5B).

In each case integrity of the RNA was tested amplifying a cDNA fragment of the human transferrin receptor.

### EXAMPLE 3

### Chromosomal Localization of the hEAG2 Gene

The chromosomal localization of the hEAG2 gene was determined by fluorescent in situ hybridization (FISH) with a biotinylated probe of a 1604 bp AatlI/BamHI restriction fragment of the hEAG2 cDNA, containing the base pairs 174 to 1777 of the open reading frame.

Under the conditions used, the hybridization efficiency was approximately 59% for the probe (among 100 checked mitotic figures, 59 of them showed signals on one pair of chromosomes). The assignment between the signal from that probe and the long arm of chromosome 14 was obtained using DAPI banding. The detailed position was further delimited based on the summary from 10 photos, whereby the hEAG2 gene is located at position 22-24 of the long arm of human chromosome 14 (14q22-24). Since this locus coincides with that of arrhythmogenic right ventricular myocardiopathy (ARVC), the gene encoding hEAG2 is likely to be responsible for this congenital disease.

### EXAMPLE 4

### Electrophysiological Properties of hEAG2

Once the complete coding sequence for hEAG2 was obtained, we characterized the electrophysiological properties of the channel by functional expression in Xenopus oocytes and in CHO cells.

For Xenopus oocyte expression, the cDNA was cloned into a suitable vector containing the translation initiation sequences and the polyadenylation sequence of Xenopus b-globin. This optimizes the expression in oocytes. The template was prepared by linearization of the construct, and RNA was synthesized using the T7 promotor. The synthetic RNA was injected into stage V-VI oocytes using standard techniques (approximately 50 ng/oocyte). The currents expressed were then measured 48-96 hours after the injection using two-electrode voltage clamp.

The current-voltage relationship of hEAG2 was determined using depolarizations lasting for 200 ms to voltages from −60 to +120 mV, from a holding potential of −100 mV. The holding potential had to be that negative due to the low threshold shown by hEAG2 (see below).

The conductance-voltage relationship was measured using tail current protocols. The oocytes were bathed in a solution containing high (115 mM) potassium concentration, and a pulse protocol analogous to the one described above was applied. In this case, due to the non-instantaneous deactivation of the current, an inward (“tail”) current can be observed upon returning to the holding potential. The amplitude of this tail current does not depend on the driving force for potassium at each test voltage (like the outward current does). Instead, it is proportional to the number of channels that were open at the time point of the return to the holding potential (that is always the same). The data points were fitted using a Boltzmann distribution. The fit gave a value for the half-activation potential (V_{half}) of −40 mV.

The depencence on the holding potential of the time constant of activation was determined using a conditioning potential between −150 and −60 mV during 5 s prior to the test pulse. The time required to achieve 80% of the maximal amplitude in the test pulse was then plotted against the conditioned potential, and fitted with a Boltzmann distribution. Extracellular magnesium is known to slow down the activation of EAG1. We determined the magnesium dependence of hEAG2 using the protocol descibed above in the presence of different external magnesium concentrations. The value of V_{half} obtained from the fit was plotted against the magnesium concentration, giving a semimaximal effect of extracellular magnesium at 80 μM (FIG. 7).

The single channel conductance of hEAG2 was estimated using non-stationary noise analysis of the current expressed in CHO-cells. The cells were transfected with a plasmid carrying the coding seuqence of hEAG2 and a chimeric protein that consists of the Zeocin resistance factor and the enhanced green fluorescence protein. Thus, the cells expressing hEAG2 can be selected by both their fluorescence and their resistance to Zeocin. The single channel conductance was estimated to be by fitting to the equation: s2=i2npo(1-po) where s2 is the current variance, i is the single channel amplitude, n de number of channel molecules in the preparation, and po is the probability that the channel is open.

The modulation of hEAG2 during cell cycle was determined using the natural cell cycle arrest of oocytes in the G2 stage of the first meiotic division. The progression of the cycle through the G2-M boundary is triggered by progesterone. We have treated the oocytes with 20 μg/ml progesterone while recording the hEAG2 currents. The treatment resulted in a reduction of the current with time. Importantly, the reduction was homogenous at all voltages tested (as opposite to what happens with EAG1).

This application incorporates by reference international application PCT/EP00/10371 (WO 01/29068), filed Oct. 20, 2000 and European application no. 99120784.6, filed Oct. 19, 1999.

1. A nucleic acid molecule comprising a nucleic acid molecule encoding a (poly)peptide having a function of the human K⁺ ion hEAG2 channel which is
(a) a nucleic acid molecule comprising a nucleic acid molecule encoding the polypeptide having the amino acid sequence of SEQ ID: No 2;
(b) a nucleic acid molecule comprising the nucleic acid molecule having the DNA sequence of SEQ ID: No 1;
(c) a nucleic acid molecule hybridizing to the complementary strand of a nucleic acid molecule of (a) or (b); or
(d) a nucleic acid molecule being degenerate to the sequence of the nucleic acid molecule of (c).

2. The nucleic acid molecule of claim 1 which is DNA.

3. The nucleic acid molecule of claim 1 which is RNA.

4. The nucleic acid molecule of any one of claims 1 to 3 encoding a fusion protein.

5. A vector comprising the nucleic acid molecule of any one of claims 1 to 4.

6. The vector of claim 5 which is an expression vector and/or a gene targeting or gene transfer vector.

7. A host transformed with a vector of claim 5 or 6.

8. The host of claim 7 which is a mammalian cell, a fungal cell, a plant cell, an insect cell or a bacterial cell.

9. A method of producing the (poly)peptide encoded by the nucleic acid molecule of any one of claims 1 to 4 comprising culturing the host of claim 7 or 8 and isolating the produced (poly)peptide.

10. A (poly)peptide encoded by the nucleic acid of any one of claims 1 to 4 or produced by the method of claim 9.

11. An antibody specifically directed to the (poly)peptide of claim 10.

12. The antibody of claim 11 which is a monoclonal antibody.

13. A pharmaceutical composition comprising the nucleic acid molecule of any one of claims 1 to 4, the vector of claim 5 or 6, the polypeptide of claim 10 and/or the antibody of claim 11 or 12 and a pharmaceutically acceptable carrier and/or diluent and/or excipient.

14. A diagnostic composition comprising the nucleic acid molecule of any one of claims 1 to 4, the vector of claim 5 or 6, the polypeptide of claim 10 and/or the antibody of claim 11 or 12.

15. A method of diagnosing tumors comprising
(a) determining the level of expression of EAG1 and hEAG2 in a sample of a subject; and
(b) diagnosing a predisposition to have a tumor if the expression level of EAG1 is aberrant, whereby the expression level of hEAG2 is normal.

16. A method for preventing or treating a disease which is caused by the undesired expression or overexpression of the nucleic acid molecule of any one of claims 1 to 3, comprising introducing an inhibitor of the expression of the nucleic acid molecule of any one of claims 1 to 3 or an inhibitor of function of the (poly)peptide of claim 10 into a mammal affected by said disease or being suspected of being susceptible to said disease.

17. A method for preventing or treating a disease which is caused by the undesired lack of expression of the nucleic acid molecule of any one of claims 1 to 3, comprising introducing a nucleic acid molecule of any one of claims 1 to 4, the vector of claim 5 or 6, the host of claim 7 or 8 or the (poly)peptide of claim 10 into a mammal affected by said disease or being suspected of being susceptible to said disease.

18. A method for preventing or treating a disease which is caused by the malfunction of the (poly)peptide of claim 10 comprising introducing an inhibitor of the expression of the nucleic acid molecule of any one of claims 1 to 3 or an inhibitor or modifying agent of the malfunction of the (poly)peptide of claim 10 or the nucleic acid molecule of any one of claims 1 to 4 encoding hEAG2 or the polypeptide of claim 10 having hEAG2 activity into a mammal affected by said disease or being suspected of being susceptible to said disease.

19. The method of claim 16 or 18 wherein said inhibitor of the expression or overexpression of said nucleic acid molecule is a nucleic acid molecule specifically hybridizing to the nucleic acid molecule of any one of claims 1 to 3.

20. The method of claim 16 or 18 wherein said inhibitor of polypeptide function is the antibody of claim 11 or 12 or a drug.

21. The method of any one of claims 16 to 20 further comprising, prior to the introduction step,
(a) obtaining cells from the mammal infected by said disease and, after said introduction step, wherein said introduction is effected into said cells; and
(b) reintroducing said cells into said mammal or into a mammal of the same species.

22. The method of any one of claims 16 to 21 wherein said cell is a germ cell, an embryonic cell or an egg cell or a cell derived therefrom.

23. A method for preventing and/or treating a congenital disease comprising introducing the nucleic acid molecule of any one of claims 1 to 4, the vector of claim 5 or 6 or a drug capable of reconstituting the function of a polypeptide of claim 10 the activity of which is blocked or diminished into a mammal affected by said disease or being susceptible to said disease.

24. A method for diagnosing a congenital disease or susceptibility to a congenital disease related to a malfunction of the polypeptide of claim 10 comprising determining a mutation in a nucleic acid sequence encoding said polypeptide.

25. The method of claim 23 or 25, wherein said congenital disease is arrythmogenic right ventricular cardiomyopathy (ARVC).

26. A method of designing a drug for the treatment of a disease which is caused by the undesired lack of expression or expression or overexpression of the nucleic acid molecule of any one of claims 1 to 3 comprising
(a) identification of a specific and potent drug;
(b) identification of the binding site of said drug by site-directed mutagenesis and chimeric protein studies;
(c) molecular modeling of both the binding site in the (poly)peptide and the structure of said drug; and
(d) modifications of the drug to improve its binding specificity for the (poly)peptide.

27. A method of identifying an inhibitor of the expression of the nucleic acid molecule of any one of claims 1 to 3 or an inhibitor of a function of the (poly)peptide of claim 10 comprising:
(a) testing a compound for the inhibition or reduction of translation wherein said compound is selected from antisense oligonucleotides and/or ribozymes; or
(b) testing a compound for the inhibition of transcription wherein said compound binds to the promoter region of the gene encoding the (poly)peptide of claim 10 and preferably with transcription factor responsive elements thereof; or
(c) testing peptides or antibodies suspected to block the proliferative activity of the (poly)peptide of claim 10 for said blocking activity.

28. The method of claim 26 or 27 wherein said drug or inhibitor is further improved by peptidomimetics or by applying phage-display or combinatorial library techniques.

29. A method of selecting a drug specifically inhibiting the expression or function of EAG1 while not effecting hEAG2 in tumor cells comprising
(a) testing a drug for inhibition of EAG1 expression or function;
(b) testing a drug for inhibition of the expression of the nucleic acid molecule of any one of claims 1 to 3 or of the function of the (poly)peptide of claim 10; and
(c) selecting a drug that tested positive in step (a) and negative in step (b).

30. A method for the production of a pharmaceutical composition comprising the steps of the method of any one of claims 26 to 29 and, furthermore, the step of formulating the drug designed or selected or the inhibitor identified in the preceding steps in a pharmaceutically acceptable form.

31. A method of inhibiting cell proliferation comprising applying an inhibitor to expression of the nucleic acid of any one of claims 1 to 3 or the (poly)peptide of claim 10.

32. A method of prognosing cancer and/or neurodegenerative diseases and/or psoriasis and/or a malfunction of the heart comprising assessing the expression of the nucleic acid molecule of any one of claims 1 to 3 or assessing the quantitative presence of the polypeptide of claim 10 in cells of a mammal.

33. The method of claim 32, wherein said cancer is mamma carcinoma or neuroblastoma or cervix carcinoma.

34. The method of claim 33, wherein said mamma carcinoma is breast adenocarcinoma, breast carcinoma ductal type.

35. The method of claim 32, wherein said neurodegenerative disease is Alzheimer's disease, Parkinson's disease, lateral amytrophic sclerosis or multiple sclerosis.

36. The method of any one of claims 16 to 23 and 32 to 35 wherein said mammal is a human, rat or mouse.

37. Use of the nucleic acid molecule of any one of claims 1 to 4 in gene therapy.

38. Kit comprising a nucleic acid molecule specifically hybridizing to the nucleic acid molecule of any one of claims 1 to 3, the vector of claim 5 or 6, the polypeptide of claim 10 and/or the antibody of claim 11 or 12.


SEQUENCE LISTING (22 sequences)

SEQ ID NO: 1 - DNA, 3289 bp, Homo sapiens
  CDS (26)..(2989)
ctggccgctgctctccagacccaggatgccggggggcaagagagggctggtggcaccgcagaacacatttttggagaacatcgtcaggcgctccagtgaatcaagtttcttactgggaaatgcccagattgtggattggcctgtagtttatagtaatgacggtttttgtaaactctctggatatcatcgagctgacgtcatgcagaaaagcagcacttgcagttttatgtatggggaattgactgacaagaagaccattgagaaagtcaggcaaacttttgacaactacgaatcaaactgctttgaagttcttctgtacaagaaaaacagaacccctgtttggttttatatgcaaattgcaccaataagaaatgaacatgaaaaggtggtcttgttcctgtgtactttcaaggatattacgttgttcaaacagccaatagaggatgattcaacaaaaggttggacgaaatttgcccgattgacacgggctttgacaaatagccgaagtgttttgcagcagctcacgccaatgaataaaacagaggtggtccataaacattcaagactagctgaagttcttcagctgggatcagatatccttcctcagtataaacaagaagcgccaaagacgccaccacacattattttacattattgtgcttttaaaactacttgggattgggtgattttaattcttaccttctacaccgccattatggttccttataatgtttccttcaaaacaaagcagaacaacatagcctggctggtactggatagtgtggtggacgttatttttctggttgacatcgttttaaattttcacacgactttcgtggggcccggtggagaggtcatttctgaccctaagctcataaggatgaactatctgaaaacttggtttgtgatcgatctgctgtcttgtttaccttatgacatcatcaatgcctttgaaaatgtggatgagggaatcagcagtctcttcagttctttaaaagtggtgcgtctcttacgactgggccgtgtggctaggaaactggaccattacctagaatatggagcagcagtcctcgtgctcctggtgtgtgtgtttggactggtggcccactggctggcctgcatatggtatagcatcggagactacgaggtcattgatgaagtcactaacaccatccaaatagacagttggctctaccagctggctttgagcattgggactccatatcgctacaataccagtgctgggatatgggaaggaggacccagcaaggattcattgtacgtgtcctctctctactttaccatgacaagccttacaaccataggatttggaaacatagctcctaccacagatgtggagaagatgttttcggtggctatgatgatggttggctctcttctttatgcaactatttttggaaatgttacaacaattttccagcaaatgtatgccaacaccaaccgataccatgagatgctgaataatgtacgggacttcctaaaactctatcaggtcccaaaaggccttagtgagcgagtcatggattatattgtctcaacatggtccatgtcaaaaggcattgatacagaaaaggtcctctccatctgtcccaaggacatgagagctgatatctgtgttcatctaaaccggaaggtttttaatgaacatcctgcttttcgattggccagcgatgggtgtctgcgcgccttggcggtagagttccaaaccattcactgtgctcccggggacctcatttaccatgctggagaaagtgtggatgccctctgctttgtggtgtcaggatccttggaagtcatccaggatgatgaggtggtggctattttagggaagggtgatgtatttggagacatcttctggaaggaaaccacccttgcccatgcatgtgcgaacgtccgggcactgacgtactgtgacctacacatcatcaagcgggaagccttgctcaaagtcctggacttttatacagcttttgcaaactccttctcaaggaatctcactcttacttgcaatctgaggaaacggatcatctttcgtaagatcagtgatgtgaagaaagaggaggaggagcgcctccggcagaagaatgaggtgaccctcagcattcccgtggaccacccagtcagaaagctcttccagaagttcaagcagcagaaggagctgcggaatcagggctcaacacagggtgaccctgagaggaaccaactccaggtagagagccgctccttacagaatggagcctccatcaccggaaccagcgtggtgactgtgtcacagattactcccattcagacgtctctggcctatgtgaaaaccagtgaatcccttaagcagaacaaccgtgatgccatggaactcaagcccaacggcggtgctgaccaaaaatgtctcaaagtcaacagcccaataagaatgaagaatggaaatggaaaagggtggctgcgactcaagaataatatgggagcccatgaggagaaaaaggaagactggaataatgtcactaaagctgagtcaatggggctattgtctgaggaccccaagagcagtgattcagagaacagtgtgaccaaaaacccactaaggaaaacagattcttgtgacagtggaattacaaaaagtgaccttcgtttggataaggctggggaggcccgaagtccgctagagcacagtcccatccaggctgatgccaagcaccccttttatcccatccccgagcaggccttacagaccacactgcaggaagtcaaacacgaactcaaagaggacatccagctgctcagctgcagaatgactgccctagaaaagcaggtggcagaaattttaaaaatactgtcggaaaaaagcgtaccccaggcctcatctcccaaatcccaaatgccactccaagtacccccccagataccatgtcaggatatttttagtgtctcaaggcctgaatcacctgaatctgacaaagatgaaatccacttttaatatatatacatatatatttgttaatatattaaaacagtatatacatatgtgtgtatatacagtatatacatatatatattttcacttgctttcaagatgatgaccacacatggattttgatatgtaaatattgcatgtccagctggattctggcctgccaaagaagatgatgattaaaaacatagatattgcttgtatattatgcagttgactgcatgcacactttacatttatttataatctctattctataataaaagagtatgatttttgttaaaaaaaaaaaaaaaaaaaaaa

SEQ ID NO: 2 - protein, 988 aa, Homo sapiens
MPGGKRGLVAPQNTFLENIVRRSSESSFLLGNAQIVDWPVVYSNDGFCKLSGYHRADVMQKSSTCSFMYGELTDKKTIEKVRQTFDNYESNCFEVLLYKKNRTPVWFYMQIAPIRNEHEKVVLFLCTFKDITLFKQPIEDDSTKGWTKFARLTRALTNSRSVLQQLTPMNKTEVVHKHSRLAEVLQLGSDILPQYKQEAPKTPPHIILHYCAFKTTWDWVILILTFYTAIMVPYNVSFKTKQNNIAWLVLDSVVDVIFLVDIVLNFHTTFVGPGGEVISDPKLIRMNYLKTWFVIDLLSCLPYDIINAFENVDEGISSLFSSLKVVRLLRLGRVARKLDHYLEYGAAVLVLLVCVFGLVAHWLACIWYSIGDYEVIDEVTNTIQIDSWLYQLALSIGTPYRYNTSAGIWEGGPSKDSLYVSSLYFTMTSLTTIGFGNIAPTTDVEKMFSVAMMMVGSLLYATIFGNVTTIFQQMYANTNRYHEMLNNVRDFLKLYQVPKGLSERVMDYIVSTWSMSKGIDTEKVLSICPKDMRADICVHLNRKVFNEHPAFRLASDGCLRALAVEFQTIHCAPGDLIYHAGESVDALCFVVSGSLEVIQDDEVVAILGKGDVFGDIFWKETTLAHACANVRALTYCDLHIIKREALLKVLDFYTAFANSFSRNLTLTCNLRKRIIFRKISDVKKEEEERLRQKNEVTLSIPVDHPVRKLFQKFKQQKELRNQGSTQGDPERNQLQVESRSLQNGASITGTSVVTVSQITPIQTSLAYVKTSESLKQNNRDAMELKPNGGADQKCLKVNSPIRMKNGNGKGWLRLKNNMGAHEEKKEDWNNVTKAESMGLLSEDPKSSDSENSVTKNPLRKTDSCDSGITKSDLRLDKAGEARSPLEHSPIQADAKHPFYPIPEQALQTTLQEVKHELKEDIQLLSCRMTALEKQVAEILKILSEKSVPQASSPKSQMPLQVPPQIPCQDIFSVSRPESPESDKDEIHF

SEQ ID NO: 3 - DNA, 29 bp, Artificial Sequence
  Description of Artificial Sequence artificial oligonucleotide sequence
ggtttccttccagaagatgtctccaaata

SEQ ID NO: 4 - DNA, 25 bp, Artificial Sequence
  Description of Artificial Sequence artificial oligonucleotide sequence
gatgacttccaaggatcctgacacc

SEQ ID NO: 5 - DNA, 27 bp, Artificial Sequence
  Description of Artificial Sequence artificial oligonucleotide sequence
ccaatgcaaaagcaggatgttcattaa

SEQ ID NO: 6 - DNA, 29 bp, Artificial Sequence
  Description of Artificial Sequence artificial oligonucleotide sequence
aatcatcctctattggctgtttgaacaac

SEQ ID NO: 7 - DNA, 29 bp, Artificial Sequence
  Description of Artificial Sequence artificial oligonucleotide sequence
taatatccttgaaagtacacaggaacaag

SEQ ID NO: 8 - DNA, 27 bp, Artificial Sequence
  Description of Artificial Sequence artificial oligonucleotide sequence
caggccaatccacaatctgggcatttc

SEQ ID NO: 9 - DNA, 21 bp, Artificial Sequence
  Description of Artificial Sequence artificial oligonucleotide sequence
ctggccgctgctctccagacc

SEQ ID NO: 10 - DNA, 27 bp, Artificial Sequence
  Description of Artificial Sequence artificial oligonucleotide sequence
tcacaaaccaagttttcagatagttca

SEQ ID NO: 11 - DNA, 25 bp, Artificial Sequence
  Description of Artificial Sequence artificial oligonucleotide sequence
agagttccaaaccattcactgtgct

SEQ ID NO: 12 - DNA, 26 bp, Artificial Sequence
  Description of Artificial Sequence artificial oligonucleotide sequence
ccagaatccagctggacatgcaatat

SEQ ID NO: 13 - DNA, 26 bp, Artificial Sequence
  Description of Artificial Sequence artificial oligonucleotide sequence
caaagcagaacaacatagcctggctg

SEQ ID NO: 14 - DNA, 29 bp, Artificial Sequence
  Description of Artificial Sequence artificial oligonucleotide sequence
ggtttccttccagaagatgtctccaaata

SEQ ID NO: 15 - DNA, 46 bp, Artificial Sequence
  Description of Artificial Sequence artificial oligonucleotide sequence
tataggtaccgaattcgcggccgccaccatgccggggggcaagaga

SEQ ID NO: 16 - DNA, 44 bp, Artificial Sequence
  Description of Artificial Sequence artificial oligonucleotide sequence
tctaggagctcgagtctagattaaaagtggatttcatctttgtc

SEQ ID NO: 17 - DNA, 27 bp, Artificial Sequence
  Description of Artificial Sequence artificial oligonucleotide sequence
accatgacaagccttacaaccatagga

SEQ ID NO: 18 - DNA, 29 bp, Artificial Sequence
  Description of Artificial Sequence artificial oligonucleotide sequence
ggtttccttccagaagatgtctccaaata

SEQ ID NO: 19 - DNA, 26 bp, Artificial Sequence
  Description of Artificial Sequence artificial oligonucleotide sequence
caaagcagaacaacatagcctggctg

SEQ ID NO: 20 - DNA, 29 bp, Artificial Sequence
  Description of Artificial Sequence artificial oligonucleotide sequence
ggtttccttccagaagatgtctccaaata

SEQ ID NO: 21 - DNA, 26 bp, Artificial Sequence
  Description of Artificial Sequence artificial oligonucleotide sequence
gtactggatagtgtggtggacgttat

SEQ ID NO: 22 - DNA, 25 bp, Artificial Sequence
  Description of Artificial Sequence artificial oligonucleotide sequence
gatgacttccaaggatcctgacacc